(19) United States
(12) Patent Application Publication
(10) Pub. No.: US 2022/0047849 A1
(43) Pub. Date: Feb. 17, 2022

(54) CATHETER WITH A PRESET CURVE

(71) Applicant: Imperative Care, Inc., Campbell, CA (US)

(72) Inventors: Brandon Yee, Oakland, CA (US); Chad C. Roue, San Jose, CA (US); Daniel Davis, Mill Valley, CA (US); Yi Yang, San Francisco, CA (US); Ashoor Shahbazi Yourgenlow, San Jose, CA (US); Jhustin Y. Scarlett, San Jose, CA (US); Miranda M. Ray, San Jose, CA (US); Farhad Khosravi, Los Altos Hills, CA (US)

(21) Appl. No.: 17/398,244

(22) Filed: Aug. 10, 2021

Related U.S. Application Data
(63) Continuation-in-part of application No. 17/343,004, filed on Jun. 9, 2021, now Pat. No. 11,207,497.
(60) Provisional application No. 63/064,270, filed on Aug. 11, 2020.

(51) Int. Cl. A61M 25/01

(52) U.S. Cl. CPC A61M 25/0152; A61M 25/0108

(57) ABSTRACT

A neurovascular catheter can have an elongate flexible tubular body. The tubular body can have a proximal end, an inclined distal end, and a side wall that defines a central lumen. The neurovascular catheter can further have a distal leading tip on a first side of the inclined distal end. The neurovascular catheter can further have a preset curve in a distal zone of the tubular body such that wherein the distal leading tip lies on a concave side of the curve.

### CROSS-REFERENCE TO RELATED APPLICATIONS

This application is a continuation-in-part of U.S. patent application Ser. No. 17/343,004, filed Jun. 9, 2021, which claims the priority benefit under 35 U.S.C. § 119(e) of U.S. Provisional Patent Application No. 63/064,270, filed Aug. 11, 2020, the entirety of each of which is hereby incorporated by reference herein.

### BACKGROUND OF THE INVENTION

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

In the context of ischemic stroke, a wide variety of thrombectomy devices have been developed to capture and retrieve a clot. These include catheters or wires which carry any of a variety of expandable cages, baskets, snares, drug or energy delivery and aspiration with or without mechanical disruption. Each of these catheters may be called upon to navigate deep into the vasculature, such as distal to the ophthalmic artery. Navigational challenges may limit the ability for many catheters to successfully reach the obstruction. Proximal retraction of the catheter may also result in tip detachment such as when the marker band engages an obstruction.

Notwithstanding the foregoing, there remains a need for new devices and methods for treating vasculature occlusions in the body, including acute ischemic stroke and occlusive cerebrovascular disease, with improved navigational abilities to traverse tortuous vasculature and reach remote treatment sites, and/or improved tensile strength to reduce the risk of tip detachment.

### SUMMARY OF THE INVENTION

There is provided in accordance with one aspect of the present invention a neurovascular catheter having a pre shaped distal tip for self-orienting with the natural curvature of a vessel to improve transvascular navigation through tortuous distal vasculature. The catheter comprises an elongate flexible tubular body, having a proximal end, an inclined distal end and a side wall defining a central lumen. A distal leading tip is carried on a first side of the inclined distal end, and a preset curve is provided in a distal zone of the tubular body. The distal leading tip lies on a concave side of the curve.

A tubular radiopaque marker may be embedded in the side wall, the tubular radiopaque marker comprising a proximal face and a distal face, wherein the distal face of the radiopaque marker inclines at an angle within a range of from about 45 degrees to about 80 degrees relative to the longitudinal axis of the central lumen.

The central lumen terminates distally in a distal port having an elliptical opening, and the elliptical opening may have an area that is at least about 105% or at least about 110% and generally within the range of from about 110% to about 125% of the cross-sectional area of the central lumen.

The elliptical opening defines an inclined distal face that inclines at an angle within a range of from about 55 degrees to about 65 degrees relative to the longitudinal axis of the central lumen.

The distal face of the radiopaque marker may also incline at an angle within the range of from about 55 degrees to about 65 degrees relative to the longitudinal axis of the central lumen. The proximal face on the radiopaque marker may be approximately perpendicular to the longitudinal axis.

The distal end of the catheter may be spaced apart from the distal face of the radiopaque marker to form an advance segment of the tubular body. The advance segment may have an axial length within a range of from about 0.1 mm to about 5 mm. The axial length of the advance segment on a leading edge side of the tubular body may be greater than the axial length of the advance segment on a trailing edge side of the tubular body. The axial length of the advance segment on the leading edge side of the tubular body may be at least about 20% longer than the axial length of the advance segment on the trailing edge side of the tubular body.

The radiopaque marker may have at least one axial slit.

The catheter may further comprise a support filament for increasing the tension resistance and/or influencing the bending characteristics in the distal zone. The support filament may comprise an axially extending filament, which may be carried between an inner liner and the helical coil, and may be positioned on the convex side of the preset curve. In one implementation, the axially extending filament may comprise Vectran.

In accordance with another aspect of the present invention, there is provided a self-orienting catheter. The catheter comprises an elongate flexible tubular body, having a proximal end, a distal zone and a side wall defining a central lumen. A tubular radiopaque marker band may be embedded in the side wall in the distal zone. The radiopaque marker band may have a first axial length measured along the side wall at a first circumferential position, and a second, longer axial length measured along the side wall at a second circumferential position offset around the circumference of the catheter by about 180 degrees from the first position; and the tubular body may have a preset curve in the distal zone. The preset curve has a concave side and a convex side, and the second, longer axial length side of the marker may be on the concave side of the curve. An axially extending filament may be positioned on the convex side.

There is also provided a catheter such as a neurovascular catheter with enhanced tensile strength, comprising an elongate flexible tubular body, having a proximal end, a distal end, and a side wall defining a central lumen; a radiopaque marker adjacent the distal end, extending at least part way around a circumference of the tubular body; and a tensile support extending axially in the side wall. The tensile support is attached to the marker to tether the marker to the catheter body to resist distal tip detachment during proximal retraction past an obstruction. In one implementation, the tensile support may extend distally along a first (e.g., inside) side of the radiopaque marker, fold around a distal edge of the radiopaque marker, and extends along a second (e.g., outside) side of the radiopaque marker.

The tensile support may comprise a plurality of fibers and in one example comprises Vectran multifilament liquid crystal polymer fiber. The tensile support may extend circumferentially at least about 180 degrees or 360 degrees or more around the marker. The sidewall of the catheter may comprise an inner liner, a tie layer and a helical coil, and the tensile support extends axially between the helical coil and the inner liner. The side wall may include an outer jacket comprising a plurality of tubular segments, a proximal tubular segment of the plurality of tubular segments having a durometer of at least about 60D, and a distal tubular segment of the plurality of tubular segments having a durometer of at most about 35D.

The radiopaque marker may comprise a proximal face and a distal face, and the distal face may incline at an angle within a range of from about 45 degrees to about 80 degrees relative to the longitudinal axis of the central lumen. The radiopaque marker may comprise an annular ring with at least one axial slit.

The catheter may comprise an inclined distal face with a distal port having an elliptical opening, and the elliptical opening may comprise an area that is at least about 105% of a transverse cross-sectional area of the central lumen. The area of the elliptical opening may be at least about 110% of the cross-sectional area of the central lumen, and the elliptical opening may lie on a plane that inclines at an angle within a range of from about 55 degrees to about 65 degrees relative to the longitudinal axis of the central lumen.

A proximal face on the radiopaque marker may be approximately perpendicular to the longitudinal axis. The distal end of the catheter may be spaced apart from the distal face of the radiopaque marker to form an advance segment of the tubular body beyond the distal end of the marker. The advance segment may have an axial length within a range of from about 0.1 mm to about 5 mm. The axial length of the advance segment on a leading edge side of the tubular body may be greater than the axial length of the advance segment on a trailing edge side of the tubular body.

The catheter may be configured to withstand at least about 1.5 pounds or at least about 3.5 pounds tension before failure (tip detachment) and in some implementations at least about 5 pounds tension before failure, or at least about 7 pounds tension before failure, in a catheter having an outside diameter of no more than about 0.10 inches or no more than about 0.080 inches.

In any of the neurovascular catheters described herein, the radiopaque marker may comprise a tubular side wall having a proximal end and a distal end, and at least one compression feature to increase the compressibility of the proximal end. The compression feature may comprises at least one compression gap in the side wall, opening at the proximal end of the sidewall and extending in a distal direction. Alternatively, the compression feature may comprise a plurality of struts joined at apexes to form a collapsible tubular side wall attached to the coil or other catheter component.

### BRIEF DESCRIPTION OF THE DRAWINGS

FIG. 1A is a side elevational view of a catheter in accordance with the present invention.

FIG. 1B is a side elevational view of a catheter with a preshaped curve in accordance with the present invention.

FIG. 1C is an enlargement of a distal section of the catheter of FIG. 1.

FIG. 1D is another enlargement of a distal section of the catheter of FIG. 1.

FIG. 2 illustrates a cross-sectional elevational view of a catheter wall according to another embodiment.

FIG. 3A illustrates a cross-sectional elevational view of a catheter wall according to another embodiment, showing one or more axially extending tension elements.

FIG. 3B describes a side elevational view of the catheter of FIG. 3A

FIG. 3C illustrates a cross-sectional view taken along the line C-C of FIG. 3B, showing one or more axially extending tension elements.

FIG. 3D is a side elevational cross section through an angled distal catheter or extension tube tip.

FIG. 3E illustrates a tip as in FIG. 3D, with a tethered marker band.

FIGS. 4A-4B are side elevational views of marker bands.

FIG. 4C is a top plan view of the marker band of FIG. 4B.

FIG. 4D is a side elevational view of an alternative marker band.

FIG. 4E is a side elevational view of an alternative marker band with an integral tubular tension element.

FIG. 4F is a side elevational view of an alternative marker band.

FIG. 4G is a side perspective view of the alternative marker band of FIG. 4F.

FIG. 4H is a side elevational view of an alternative marker band.

FIG. 4I is a side perspective view of the alternative marker band of FIG.

FIG. 5A illustrates a side elevational view of a progressively enhanced flexibility catheter according to an embodiment.

FIG. 5B is a proximal end view of the enhanced flexibility catheter of FIG. 5A.

### DETAILED DESCRIPTION OF THE PREFERRED EMBODIMENT

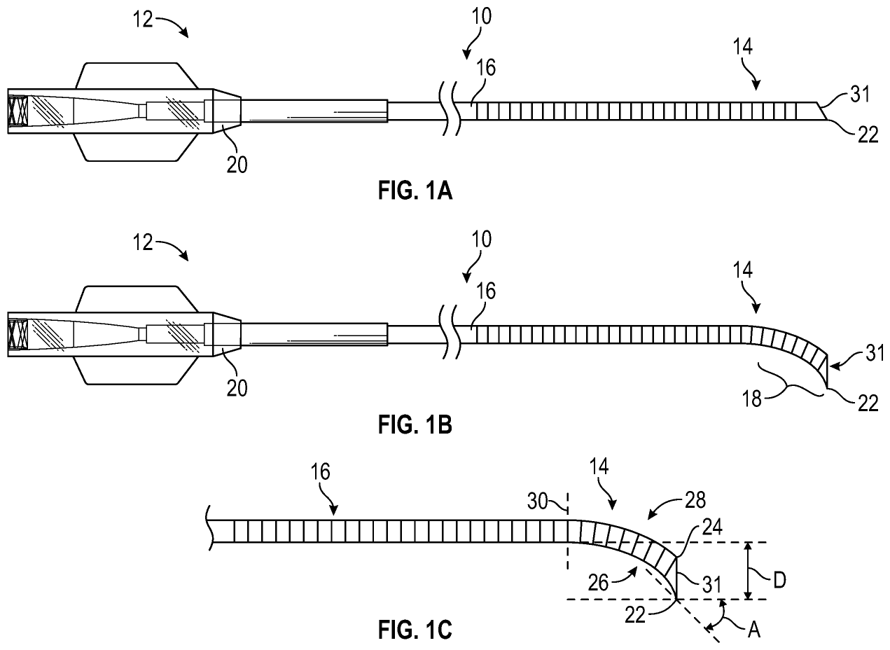

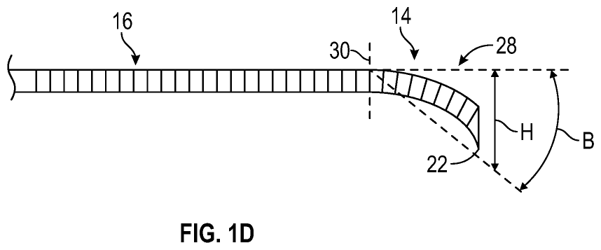

Referring to FIG. 1A-1C, there is disclosed a catheter 10 in accordance with one aspect of the present invention. Although primarily described in the context of an aspiration catheter with a single central lumen, catheters of the present invention can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the present invention will be described primarily in the context of removing obstructive material from remote vasculature in the brain, but has applicability as an access catheter for delivery and removal of any of a variety of diagnostics or therapeutic devices with or without aspiration.

The catheters disclosed herein may readily be adapted for use throughout the body wherever it may be desirable to distally advance a low profile high flexibility catheter into small and/or tortuous vasculature. For example, catheter shafts in accordance with the present invention may be dimensioned for use throughout the coronary and peripheral vasculature, the gastrointestinal tract, the urethra, ureters, Fallopian tubes and other lumens and potential lumens, as well. The catheter shaft construction of the present invention may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants.

The catheter 10 generally comprises an elongate tubular body 16 extending between a proximal end 12 and a distal functional end 14. The catheter 10 may have no preset curve (FIG. 1A) or may have a preset curve (FIGS. 1B-1C). The length of the tubular body 16 depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site, as discussed in further detail below.

Catheters in accordance with the present invention will have a length and diameter suitable for the intended access point and target location. In one example, referring to FIGS. 1A-1C, the catheter 10 may have an effective length from the manifold or hub 20 to distal tip 22 generally no more than about 180 cm or no more than about 160 cm. and typically from about 70 cm to about 150 cm, from about 90 cm to about 130 cm, or from about 105 cm to about 115 cm. The outer diameter of the catheter 10 may be from about 0.035 inches to about 0.15 inches, from about 0.09 inches to about 0.13 inches, and may be lower in a distal segment than in a proximal segment.

The inner diameter of the catheter 10 in a single central lumen embodiment may be greater than or equal to about 0.1 inches, greater than or equal to about 0.088 inches, or greater than or equal to about 0.08 inches, or greater than or equal to about 0.06. The inner diameter of the catheter 10 in a single central lumen embodiment may be less than about 0.20 inches or 0.15 inches, or less than or equal to about 0.11 inches, less than or equal to about 0.1 inches, less than or equal to about 0.088 inches, or less than or equal to about 0.07 inches, and often no more than about 0.095 inches.

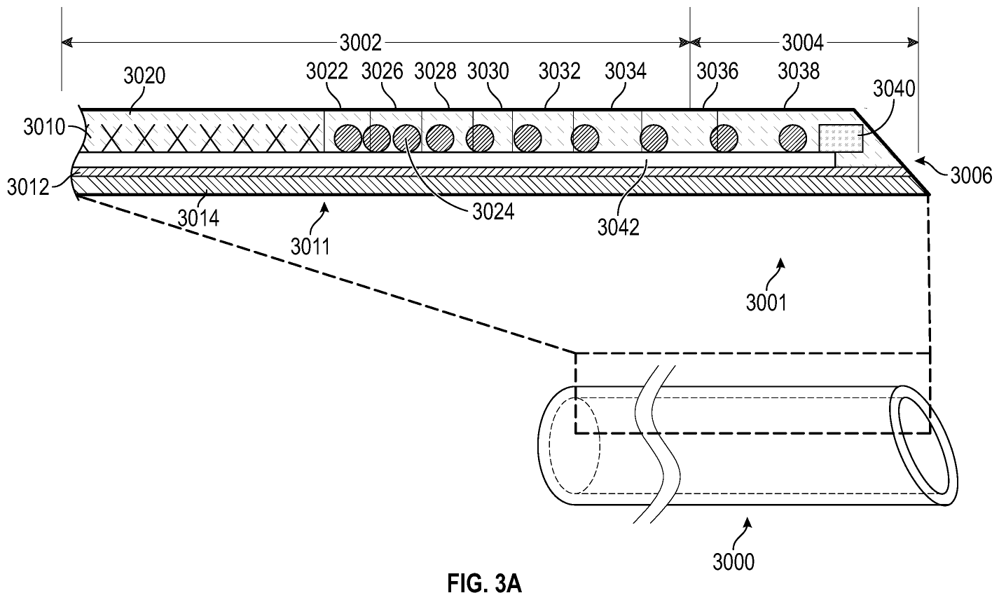

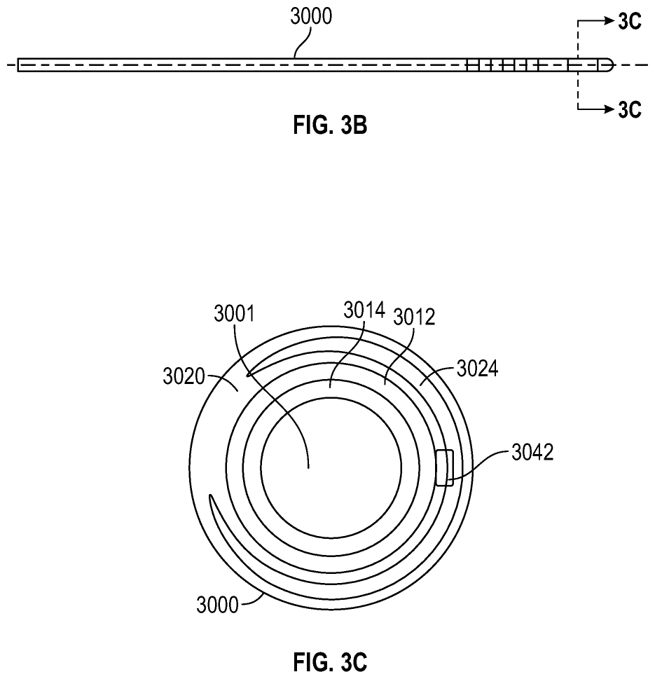

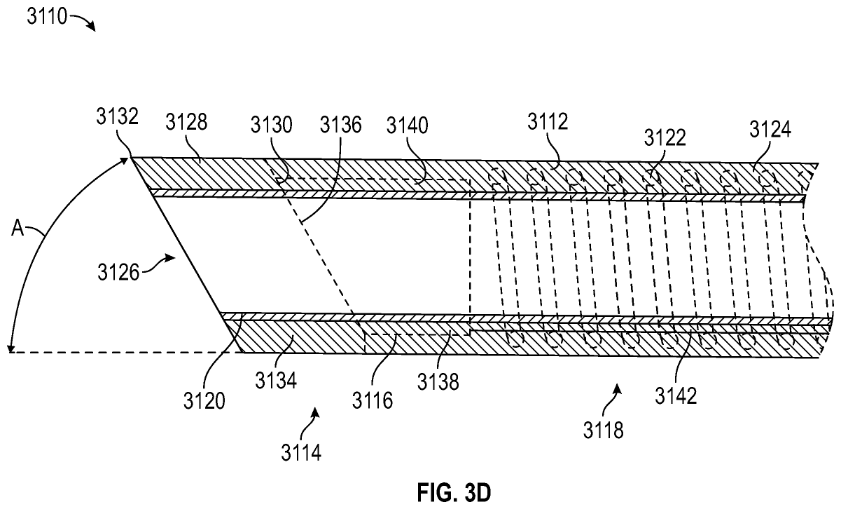

FIG. 1C illustrates a distal section of the tubular body 16. A passive distal steering zone 18 on the tubular body 16 is provided with a pre-shaped curve, having a concave side 26 and a convex side 28. The tubular body 16 is additionally provided with an inclined face 31, discussed in greater detail in connection with FIG. 3D. The inclined face 31 produces a leading edge at distal tip 22, and an opposing trailing edge 24. The leading edge 22 is disposed on the concave side 26 of the pre-shaped curve.

In an unconstrained configuration, the pre-shaped curve establishes an angle A between the longitudinal axis of the tubular body 16 proximally of the curve (i.e., concave side of the pre-shaped curve), and the longitudinal axis of the distal most 2 or 3 mm of the tubular body 16. Angle A is generally within the range of from about 25° to about 55°, preferably no more than about 50°, and in some implementations between about 30° and about 40°. The angle A is preferably within the range of from about 32° and about 38°, and in one example is about 35°. Additionally, in some implementations, angle A is within the range of from about 25° to about 45°, about 20° to about 45°, about 15° to about 55°, or about 15° to about 50°. The angle is low enough that in combination with the low lateral bias of the preset curve, the tip 22 of the catheter will follow the native vasculature but not penetrate the side wall into extravascular space.

In some embodiments, as shown in FIG. 1D, the pre-shaped curve establishes an angle B between the longitudinal axis of the tubular body 16 proximally of the curve (e.g., at transition 30 or at convex side of the pre-shaped curve or based on proximal edge of markerband), and the distal tip 22. Angle B is generally within the range of from about 25° to about 55°, preferably no more than about 50°, and in some implementations between about 25° and about 35°. The angle A is preferably within the range of from about 27° and about 33°, and in one example is about 30°. Additionally, in some implementations, angle B is within the range of from about 25° to about 45°, about 20° to about 45°, about 15° to about 55°, or about 15° to about 50°.

In some embodiments, as further shown in FIG. 1, the pre-shaped curve establishes a height H between the longitudinal axis of the tubular body 16 proximally of the curve (e.g., at transition 30 or at convex side of the pre-shaped curve or based on proximal edge of markerband), and the distal tip 22. Height H is generally within the range of from about 0.25 cm to about 0.6 cm, and in some implementations, between about 0.3 cm to about 0.4 cm or about 0.35 cm to about 0.45 cm or about 0.4 cm to about 0.5 cm or about 0.45 cm to about 0.55 cm.

The lateral limit of unconstrained deflection D is generally within the range of from about 0.1 and about 0.2 inches and, in one embodiment, is about 0.15 inches. In some implementations, unconstrainted deflection D is generally within the range of from about 0.05 to about 0.15 inches, about 0.06 inches to about 0.13 inches, about 0.07 inches to about 0.12 inches, about 0.075 inches to about 0.12 inches, etc. Unconstrained deflection D will typically be no greater than about 0.3 inches or about 0.25 inches or about 0.2 inches, depending upon the catheter diameter.

The tubular body 16 includes a transition 30 at the proximal limit of the pre-shaped curve. The arc length of the pre shaped curve measured from the transition 30 to the distal tip 22 is generally less than about 2 cm or less than about 1.5 cm or less than about 1 cm, and often between about 0.5 cm and about 1.5 cm and in some implementations the length is about 1.1 cm to about 1.4 cm. In some implementations, the arc length is within the range of from about 0.75 cm to about 1.75 cm or about 0.5 cm to about 2 cm or about 1.25 cm to about 2 cm.

The tubular body 16 of the present invention is sufficiently flexible that the catheter is trackable for ease of advancement through even narrow and/or tortuous body passageways. The catheter may bend in any plane in three-dimensional space, in response to advancing through the curvature of the vasculature. Thus, at least the distal preshaped curve may spontaneously twist about the longitudinal axis of the catheter during advancement through a body passageway as itself orients to follow the lowest energy state configuration through the curvature of the vessel. Torque transmission through the tubular body 16 is sufficiently low (low torsional stiffness) that the distal end of the catheter is able to twist about its axis as desired in both clockwise and counterclockwise directions to self-orient with the vasculature during distal advance, without needing the proximal end of the catheter to rotate. In some embodiments, the distal end of the catheter can twist at least about 10 degrees, at least about 20 degrees or in some implementations at least about 45 degrees or 90 degrees or more in either direction without any rotation of the proximal end of the catheter. The self-orientation or twisting of the catheter may optimize an angle of interaction of the distal end of the catheter with a clot to improve or maximize clot ingestion.

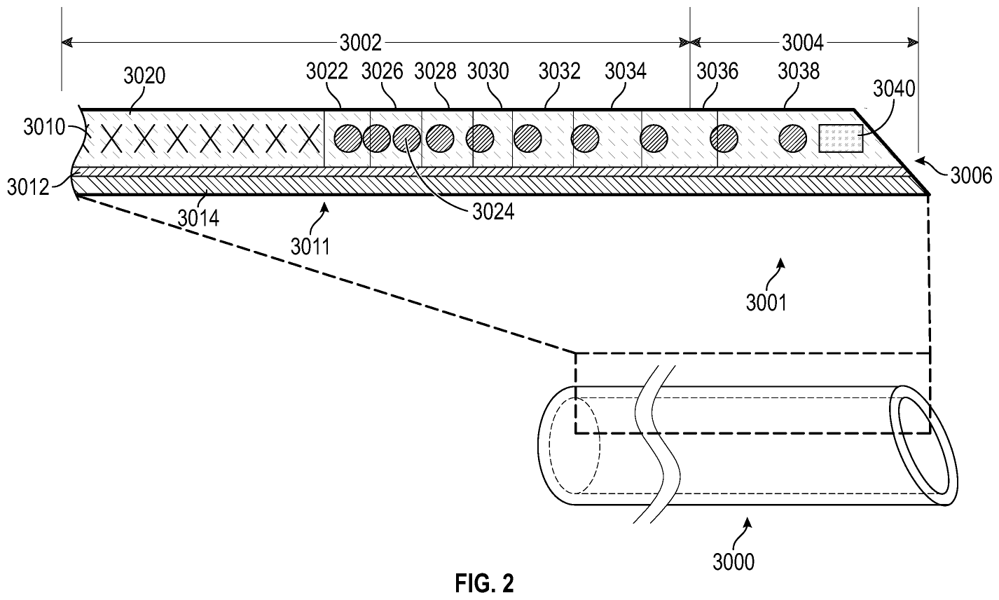

FIG. 2 illustrates a cross section through the sidewall of a distal portion of a single lumen catheter, that may be formed either with or without the preset curve. Adjacent loops or filars of the coil 3024 may have a constant pitch throughout the length of the coil or may be closely tightly wound in a proximal zone with a distal section having looser spacing between adjacent loops. In an embodiment having a coil section 3024 with an axial length of at least between about 20% and about 30% of the overall catheter length, (e.g., 28 cm coil length in a 110 cm catheter shaft 16), at least the distal about 1 cm or about 2 cm or about 3 cm or about 4 cm of the coil will have a spacing that is at least about 130%, and in some implementations at least about 150% or more than the spacing in the proximal coil section. In a 110 cm catheter shaft 3000 having a Nitinol coil, the spacing in the proximal coil may be about 0.004 inches and in the distal section may be at least about 0.006 inches or about 0.007 inches or more.

The distal end of the coil 3024 can be spaced proximally from the distal end of the inner liner 3014, for example, to provide room for an annular radiopaque marker 3040. The coil 3024 may be set back proximally from the distal end, in some embodiments, by approximately no more than about 1 cm, about 2 cm, or about 3 cm. In one embodiment, the distal end of the catheter 10 is provided with a beveled distal surface 3006 residing on a plane having an angle of at least about 10 degrees or about 20 degrees and in one embodiment about 30 degrees with respect to a longitudinal axis of the catheter 10. The radiopaque marker 3040 may reside in a plane that is transverse to the longitudinal axis. Alternatively, at least the distally facing edge of the annular radiopaque marker 3040 may be an ellipse, residing on a plane which is inclined with respect to the longitudinal axis to complement the bevel angle of the distal surface 3006. Additional details are described in connection with FIG. 3D below.

After applying the proximal braid 3010 over tie layer 3012, the distal coil 3024 and the RO marker 3040 are provided with an outer jacket 3020 such as a shrink wrap tube to enclose the catheter body 16. The outer shrink-wrapped sleeve 3020 may comprise any of a variety of materials, such as polyethylene, polyurethane, polyether block amide (e.g., PEBAX™), nylon or others known in the art. Sufficient heat is applied to cause the polymer to flow into and embed the proximal braid and distal coil.

In one implementation, the outer shrink wrap jacket 3020 is formed by sequentially advancing a plurality of short tubular segments 3022, 3026, 3028, 3030, 3032, 3034, 3036, 3038 concentrically over the catheter shaft subassembly, and applying heat to shrink the sections on to the catheter 10 and provide a smooth continuous outer tubular body. The foregoing segmented construction may extend along at least the most distal about 10 cm, and preferably at least about the most distal about 20 cm, about 25 cm, about 30 cm, about 35 cm, about 40 cm, or more than about 40 cm of the catheter body 10. The entire length of the outer shrink wrap jacket 3020 may be formed from tubular segments and the length of the distal tubular segments (e.g., 3022, 3026, 3028, 3030, 3032, 3034, 3036, 3038) may be shorter than the one or more tubular segments forming the proximal portion of the outer shrink wrap jacket 3020 in order to provide proximal backup support and steeper transitions in flexibility toward the distal end of the catheter 10.

The durometer of the outer wall segments may decrease in a distal direction. For example, proximal segments such as 3022 and 3026, may have a durometer of at least about 60D or about 70D, with gradual decrease in durometer of successive segments in a distal direction to a durometer of no more than about 35D or about 25D or lower. A 25 cm section may have at least about 3 or about 5 or about 7 or more segments and the catheter 10 overall may have at least about 6 or about 8 or about 10 or more distinct flexibility zones. The distal 1 or 2 or 4 or more segments 3036, 3038, may have a smaller OD following shrinking than the more proximal segments 3022-3034 to produce a step down in OD for the finished catheter body 16. The length of the lower OD section 3004 may be within the range of from about 3 cm to about 15 cm and, in some embodiments, is within the range of from about 5 cm to about 10 cm such as about 7 cm or about 8 cm, and may be accomplished by providing the distal segments 3036, 3038 with a lower wall thickness.

In another embodiment, the most distal portion of the catheter 10 may comprise a durometer of less than approximately 35D (e.g., 25D) to form a highly flexible distal portion of the catheter and have a length between approximately 25 cm and approximately 35 cm. The distal portion may comprise one or more tubular segments of the same durometer (e.g., segment 3038). A series of proximally adjacent tubular segments may form a transition region between a proximal stiffer portion of the catheter 3000 and the distal highly flexible portion of the catheter. The series of tubular segments forming the transition region may have the same or substantially similar lengths, such as approximately 1 cm.

The relatively short length of each of the series of tubular segments may provide a steep drop in durometer over the transition region. For example, the transition region may have a proximal tubular segment 3036 (proximally adjacent the distal portion) having a durometer of approximately 35D. An adjacent proximal segment 3034 may have a durometer of approximately 55D. An adjacent proximal segment 3032 may have a durometer of approximately 63D. An adjacent proximal segment 3030 may have a durometer of approximately 72D.

More proximal segments may comprise a durometer or durometers greater than approximately 72D and may extend to the proximal end of the catheter or extension catheter segment. For instance, an extension catheter segment may comprise a proximal portion greater than approximately 72D between about 1 cm and about 3 cm. In some embodiments, the proximal portion may be about 2 cm long. In some embodiments, the most distal segments (e.g., 3038-3030) may comprise PEBAX™ and more proximal segments may comprise a generally stiffer material, such as Vestamid®.

The inner diameter of the catheter 10 may be between approximately 0.06 and 0.08 inches, between approximately 0.065 and 0.075 inches, or between approximately 0.068 and 0.073 inches. In some embodiments, the inner diameter is approximately 0.071 inches.

In some embodiments, the distal most portion may taper to a decreased inner diameter as described elsewhere herein. The taper may occur approximately between the distal highly flexible portion and the transition region (e.g., over the most proximal portion of the distal highly flexible portion). The taper may be relatively gradual (e.g., occurring over approximately 10 or more cm) or may be relatively steep (e.g., occurring over less than approximately 5 cm). The inner diameter may taper to an inner diameter between about 0.03 and about 0.06 inches. For example, the inner diameter may be about 0.035 inches, about 0.045 inches, or about 0.055 inches at the distal end of the catheter 3000. In some embodiments, the inner diameter may remain constant, at least over the catheter extension segment.

In some embodiments, the coil 3024 may extend proximally from a distal end of the catheter 10 along the highly flexible distal portion ending at the distal end of the transition region. In other embodiments, the coil 3024 may extend from a distal end of the catheter to the proximal end of the transition region, to a point along the transition region, or proximally beyond the transition region. In other embodiments, the coil 3024 may extend the entire length of the catheter 10 or catheter extension segment as described elsewhere herein. The braid 3010, when present, may extend from the proximal end of the coil 3024 to the proximal end of the catheter 10.

Referring to FIGS. 3A-3D, the catheter may further comprise an axial tension element or support such as a ribbon or one or more filaments or fibers for increasing the tension resistance and/or influencing the bending characteristics in the distal zone. The tension support may comprise one or more axially extending mono strand or multi strand filaments 3042. The one or more tension element 3042 may be axially placed inside the catheter wall near the distal end of the catheter. The filament may be positioned on the convex side of a catheter having the preset curve. The one or more tension element 3042 may serve as a tension support and resist elongation of the catheter wall under tension (e.g., when the catheter is being proximally retracted through tortuous or narrowed vasculature).

At least one of the one or more tension element 3042 may proximally extend along the length of the catheter wall from within about 1.0 cm from the distal end of the catheter to less than about 10 cm from the distal end of the catheter, less than about 20 cm from the distal end of the catheter, less than about 30 cm from the distal end of the catheter, less than about 40 cm from the distal end of the catheter, or less than about 50 cm from the distal end of the catheter.

The one or more tension element 3042 may have a length greater than or equal to about 40 cm, greater than or equal to about 30 cm, greater than or equal to about 20 cm, greater than or equal to about 10 cm, or greater than or equal to about 5 cm.

At least one of the one or more tension element 3042 may extend at least about the most distal 50 cm of the length of the catheter, at least about the most distal 40 cm of the length of the catheter, at least about the most distal 30 cm or about 20 cm or about 10 cm of the length of the catheter.

In some implementations, the tension element extends proximally from the distal end of the catheter along the length of the coil 24 and ends proximally within about 5 cm or about 2 cm or less either side of the transition 3011 between the coil 3024 and the braid 3010. The tension element may end at the transition 3011, without overlapping with the braid 3010.

The one or more tension element 3042 may be placed near or radially outside the tie layer 3012 or the inner liner 3014. The one or more tension element 3042 may be placed near or radially inside the braid 3010 and/or the coil 3024. The one or more tension element 3042 may be carried between the inner liner 3014 and the helical coil 3024, and may be secured to the inner liner or other underlying surface by an adhesive prior to addition of the next outer adjacent layer such as the coil.

When more than one tension element 3042 or filament bundles are spaced circumferentially apart in the catheter wall, the tension elements 3042 may be placed in a radially symmetrical manner. For example, the angle between two tension elements 3042 with respect to the radial center of the catheter may be about 180 degrees. Alternatively, depending on desired clinical performances (e.g., flexibility, trackability), the tension elements 3042 may be placed in a radially asymmetrical manner. The angle between any two tension elements 3042 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 3042 may comprise materials such as Vectran, Kevlar, Polyester, Meta-Para-Aramide, or any combinations thereof. At least one of the one or more tension element 3042 may comprise a single fiber or a multi-fiber bundle, and the fiber or bundle may have a round or rectangular (e.g., ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 3042, as measured in the radial direction, may be no more than about 2%, 5%, 8%, 15%, or 20% of that of the catheter 10.

The cross-sectional dimension of the one or more tension element 3042, as measured in the radial direction, may be no more than about 0.001 inches, no more than about 0.002 inches, no more than about 0.004 inches, no more than about 0.006 inches, no more than about 0.008 inches, or about 0.015 inches.

The one or more tension element 3042 may increase the tensile strength of the distal zone of the catheter before failure under tension to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

Any of the catheters disclosed herein, whether or not an axial tension element is included, may be provided with an angled distal tip. Referring to FIG. 3D, distal catheter tip 3110 comprises a tubular body 3112 which includes an advance segment 3114, a marker band 3116 and a proximal segment 3118. An inner tubular liner 3120 may extend throughout the length of the distal catheter tip 3110, and may comprise dip coated PTFE.

A reinforcing element 3122 such as a braid or spring coil is embedded in an outer jacket 3124 which may extend the entire length of the distal catheter tip 3110.

The advance segment 3114 terminates distally in an angled face 3126, to provide a leading side wall portion 3128 having a length measured between the distal end 3130 of the marker band 3116 and a distal tip 3132. A trailing side wall portion 3134 of the advance segment 3114, has an axial length in the illustrated embodiment of approximately equal to the axial length of the leading side wall portion 3128 as measured at approximately 180 degrees around the catheter from the leading side wall portion 3128. The leading side wall portion 3128 may have an axial length within the range of from about 0.1 mm to about 5 mm and generally within the range of from about 1 to 3 mm. The trailing side wall portion 3134 may be at least about 0.1 or 0.5 or 1 mm or 2 mm or more shorter than the axial length of the leading side wall portion 3128, depending upon the desired performance.

The angled face 3126 inclines at an angle A within the range of from about 45 degrees to about 80 degrees from the longitudinal axis of the catheter. For certain implementations, the angle is within the range of from about 55 degrees to about 65 degrees or within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105%, and no more than about 130%, in some implementations within the range of from about 110% and about 125% and in one example is about 115%.

In the illustrated embodiment, the axial length of the advance segment is substantially constant around the circumference of the catheter, so that the angled face 3126 is approximately parallel to the distal surface 3136 of the marker band 3116. The marker band 3116 has a proximal surface approximately transverse to the longitudinal axis of the catheter, producing a marker band 3116 having a right trapezoid configuration in a side elevational view. A short sidewall 3138 is rotationally aligned with the trailing side wall portion 3134, and has an axial length within the range of from about 0.2 mm to about 4 mm, and typically from about 0.5 mm to about 2 mm. An opposing long sidewall 3140 is rotationally aligned with the leading side wall portion 3128. Long sidewall 3140 of the marker band 3116 is generally at least about 10% or 20% longer than short sidewall 3138 and may be at least about 50% or 70% or 90% or more longer than short sidewall 3138, depending upon desired performance. Generally, the long sidewall 3140 will have a length of at least about 0.5 mm or 1 mm and less than about 5 mm or about 4 mm.

Any of the marker bands described herein may be a continuous annular structure, or may optionally have at least one and optionally two or three or more axially extending slits 3117 throughout its length. The slit may be located on the short sidewall 3138 or the long sidewall 3140 or in between, depending upon desired bending characteristics. Any of the marker bands described herein may comprise any of a variety of radiopaque materials, such as a platinum/iridium alloy, with a wall thickness preferably no more than about 0.003 inches and in one implementation is about 0.001 inches. In one implementation, at least one axial slit is aligned with the convex side of the preset curve, and the filament extends distally beyond the proximal face of the marker and into the axial slit.

The marker band zone of the assembled catheter may have a relatively high bending stiffness and high crush strength, such as at least about 50% or at least about 100% less than proximal segment 18 but generally no more than about 200% less than proximal segment 3118. The high crush strength may provide radial support to the adjacent advance segment 3114 and particularly to the leading side wall portion 3128, to facilitate the functioning of distal tip 3132 as an atraumatic bumper during transluminal advance and to resist collapse under vacuum. The proximal segment 3118 preferably has a lower bending stiffness than the marker band zone, and the advance segment 3114 preferably has even a lower bending stiffness and crush strength than the proximal segment 3118.

The advance segment 3114 may comprise a distal extension of the outer jacket 3124 and optionally the inner liner 3120, without other internal supporting structures distally of the marker band 3116. Outer jacket may comprise extruded Tecothane. The advance segment 3114 may have a bending stiffness and radial crush stiffness that is no more than about 50%, and in some implementations no more than about 25% or 15% or 5% or less than the corresponding value for the proximal segment 3118.

A tension element 3142 with dimensions and materials as has been discussed elsewhere herein extends through at least a distal portion of the length of the proximal segment 3118. As illustrated, the tension element 3142 may terminate distally at a proximal surface of the marker band 3116 and extend axially radially outwardly of the tubular liner 3120 and radially inwardly from the support coil 3122. Alternatively, the marker band may be provided with at least one or two axially extending slits 3117, and the fiber can extend into the slit, thus axially overlapping with the marker band. Tension element 3142 may extend substantially parallel to the longitudinal axis, or may be inclined into a mild spiral having no more than 10 or 7 or 3 or 1 or less complete revolutions around the catheter along the length of the spiral. The fiber may comprise a high tensile strength material such as a multifilament yarn spun from liquid crystal polymer such as a Vectran multifilament LCP fiber.

In the implementation illustrated in FIG. 3E, the tension element 3142 extends axially in a distal direction along the outside of (or inside of) the coil, towards an anchor which may be in the form of a continuous or slit annular ring such as the marker band 3116 which may have an inclined distal face as has been discussed. The tension element 3142 is preferably secured to the anchor, to increase the tensile force threshold before failure by tip detachment. This allows the catheter to be pulled proximally through restrictions such as a vascular restriction or a kink in the guide catheter which may collapse but not detach the marker band. The enhanced tensile strength also provides tactile feedback to the physician when they encounter a restriction that may shear off the marker band. In an implementation having a slit 3117, the axis of the tension element 3142 may be circumferentially offset from the slit 3117 to avoid the fiber pulling through the slit.

The tension element 3142 may be secured to the anchor in any of a variety of ways, depending upon the structures and materials involved, including adhesives, welding or mechanical interference fit. In the illustrated implementation, the tension element 3142 is wrapped around at least a distally facing edge of the marker band 3116, such as the distal edge of the marker band or a proximal edge of an aperture through the marker band 3116. In one implementation, the tension element 3142 extends axially along a first surface of the marker band beyond the marker band and is folded back around the distal edge of the marker band, and onto a second surface of the marker band and secured to the tubular body (e.g., to the marker band, or to itself).

In the illustrated example, a first segment 3150 of the tension element 3142 extends axially along the catheter body over or preferably under the coil, under the marker band 3116 and distally along the inside surface of the marker band to the distal edge 3156 of the marker band. The tension element is folded back over the distal edge 3156 and extends proximally over the outside surface of the marker band along an angled segment 3152 and wrapped in a circumferential direction around the tubular body such as over the marker band 3116 and/or adjacent catheter side wall to an end 3154. The tension element may be wrapped circumferentially around through an angle of at least about 180 degrees and preferably at least about 270 degrees or about 360 degrees or at least about 450 degrees or more. The tension element may be tacked down over the marker band or adjacent catheter shaft with an adhesive such as Loctite prior to applying the outer polymer jacket.

Alternatively, the tension element may be folded around the marker band and back proximally over itself, running proximally for a bonding zone of at least about 1 or about 2 or about 5 or more cm along which it may be bonded to itself before being encased in the outer jacket.

In the illustrated implementation, the tension element crosses the marker band at a point within the range of approximately 20 degrees to about 40 degrees circumferentially offset from the center of the slit 3117. Alternatively, the tensile element may cross the marker band within the range of approximately 80 degrees to about 100 degrees offset from the slit, or within the range of from about 170 degrees to about 190 degrees from the slit. In an implementation in which the slit is not located at the shortest axial dimension of the marker band, the foregoing offsets may be measured from the shortest axial dimension.

The radial compressibility of the marker band may desirably increase in the proximal direction from the distal end of the marker band to the proximal end of the marker band, to form a continuous or stepped graduated compressibility. This may facilitate radial compressibility at the proximal end of the marker band such as when the marker band encounters an obstruction (e.g., vascular obstruction or kink in the guide catheter) during proximal retraction of the catheter. Further proximal retraction allows the side wall of the marker band to ramp up to the diameter of the distal end of the marker band, displacing the obstruction laterally and/or progressively collapsing the marker band to allow the marker band to squeeze past the obstruction. This, in combination with the attached tensile element, optimizes the likelihood of avoiding marker band detachment.

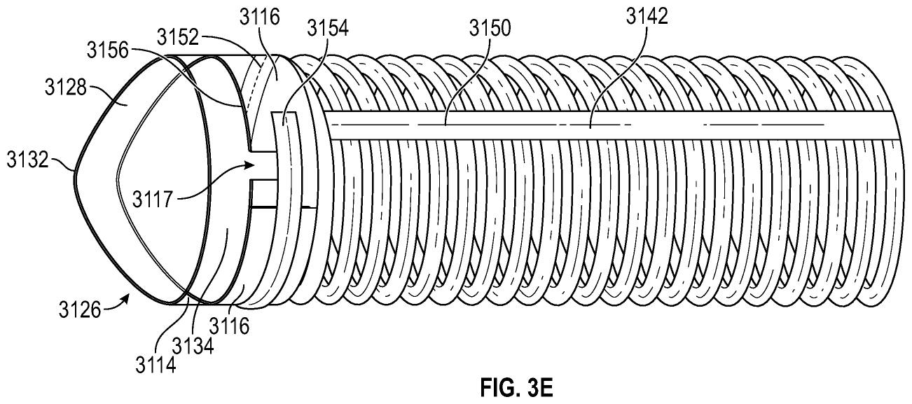

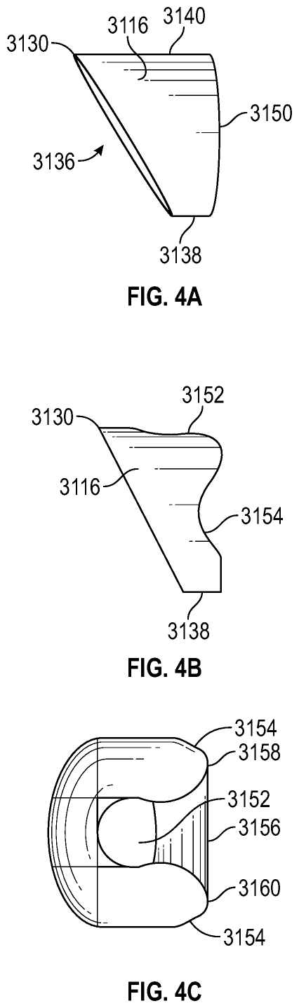

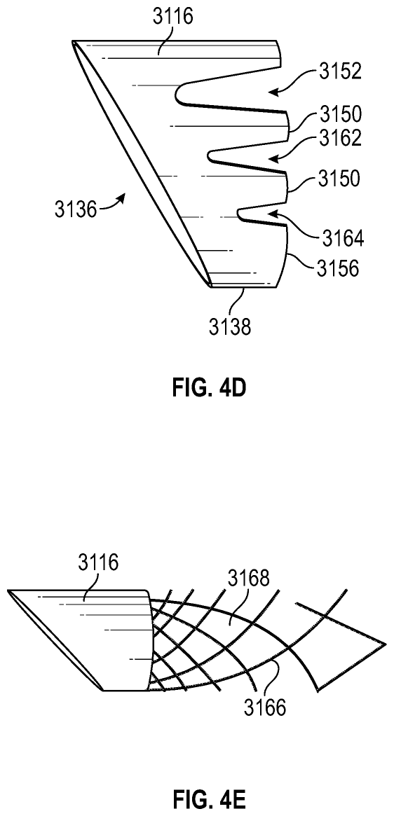

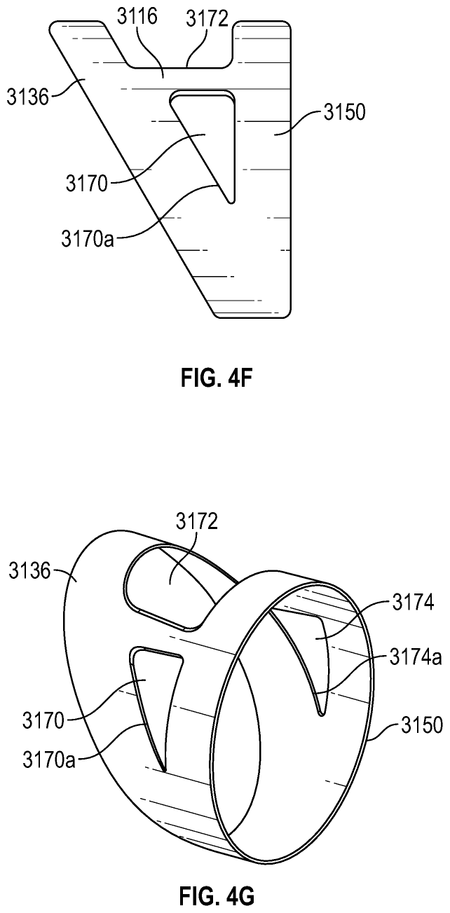

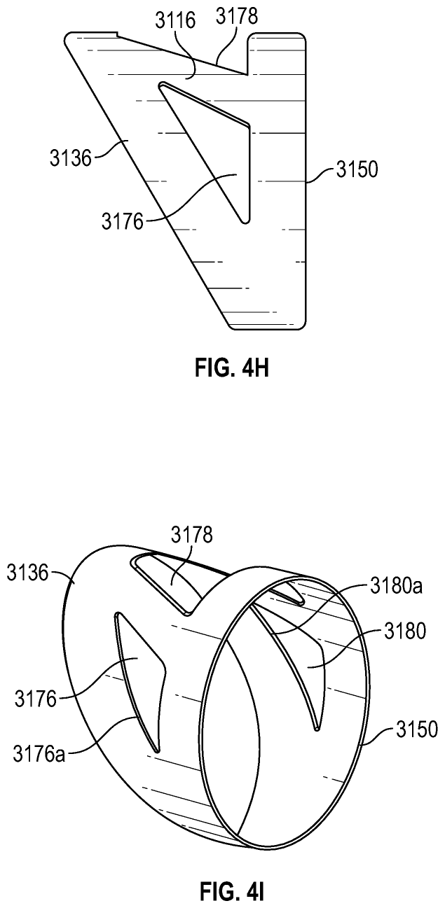

The basic geometry of a previously described marker band 3116 is illustrated in FIG. 4A. Marker band 3116 extends between a proximal transverse face 3150 and a distal inclined face 3136. A long side wall 3140 terminates distally in a distal tip 3130. An opposing short side wall 3138 may contain an axial slit as has been discussed.

Referring to FIG. 4B, a marker band 3116 is provided with a compression feature that increases the radial compressibility of the proximal end of the marker band. In the illustrated implementation, the compression feature comprises at least a first compression gap 3152 and may comprise at least a second compression gap in the form of a proximal facing concavity 3154. The first compression gap 3152 extends distally from the proximal face 3150 at least about 25% and in some implementations at least about 50% or about 70% or more of the length of long sidewall 3140.

The second compression gap may extend distally from the proximal face 3150, rotated approximately 90 degrees in a first circumferential direction from the first compression gap 3152. At least a third compression gap may be provided, rotated about 90 degrees in a second circumferential direction from the first compression gap 3152.

The foregoing construction provides an arcuate base 3156 in the form of the proximal edge of the marker band 3116, lying on the plane of proximal face 3150, for contacting the distal end of the coil or other sidewall reinforcement in the catheter body. A first foot 3158 and a second foot 3160 are also formed, also lying approximately on the plane corresponding to proximal face 3150, for supporting the marker band 3116 against the distal end of the spring coil or other catheter body reinforcement. This allows radial compression of the proximal end of the marker band 3116, while also supporting the marker band 3116 against tilting relative to the distal face of the coil.

In the implementation shown in FIG. 4D, marker band 3116 having the characteristics of the marker band of FIG. 4A is modified by providing at least a first compression gap 3152 that facilitates radial compression. A second compression gap 3162 and optionally a third compression gap 3164 or more may be provided depending upon desired performance. The proximal openings of the compression gaps may reside on a transverse plane, such as the proximal face 3150 of marker band 3116. Each compression gap preferably has a width measured in a circumferential direction at the proximal end that exceeds the width near the distal end of the compression gap. The axial depth of the compression gaps may be approximately equal, so that the distal ends of the compression gaps all align in a transverse plane that is approximately parallel with the proximal face 3150. Alternatively, as illustrated in FIG. 4D, the distal ends of the compression gaps may be aligned progressively such that they lie on an inclined plane that may be approximately parallel to the inclined distal face 3136.

Alternatively, as shown in the marker bands of FIGS. 4F-4I, one or more compression gaps may extend between a proximal transverse face 3150 and a distal inclined face 3136, but not intersect the proximal transverse face 3150 (in contrast, for example, to the marker band of FIG. 4E). As described above, each compression gap 3176, 3178, 3180 may have a width measured in a circumferential direction at the proximal end that exceeds the width near the distal end of the compression gap, as shown in FIGS. 4H-4I. Alternatively, each compression gap 3170, 3172, 3174 may have a width measured in a circumferential direction at the proximal end that substantially equals or is substantially similar to the width near the distal end of the compression gap, as shown in FIGS. 4F-4G. An angle and/or shape of the distal end 3170a, 3174a, 3176a, 3180a of compression gaps 3170, 3174, 3176, 3180, respectively, may substantially equal or be similar to the angle of the distal face 3136, as described above and shown in FIGS. 4F-4I. The circumferentially continuous proximal transverse face 3150 of each of the embodiments in FIGS. 4F-4I is such that it enables securing to the coil, as described elsewhere herein and below.

In addition to or as an alternative to the tension element, any of the marker bands disclosed herein may be secured to the coil such as by adhesives, welding, or mechanical interference fit. In one mechanical interference fit implementation, a helical slot may be formed in the proximal sidewall of the marker band, extending circumferentially through at least about 45 degrees, and in some implementations at least about 180 degrees or about 360 degrees or more. This allows the distal end of the helical coil to be screwed into the helical slot in the marker band side wall while preserving the ID of the lumen and OD of the catheter across the joint.

As a further alternative, one or more tension elements maybe integrally formed with the marker band, such as by laser cutting the marker band and an elongate, proximally extending axial or helical strut tension element from a single tube stock.

The tension element may take the form of at least one and optionally at least two or four or 10 or more struts, which may extend proximally in a linear, spiral, or intersecting e.g., diamond pattern.

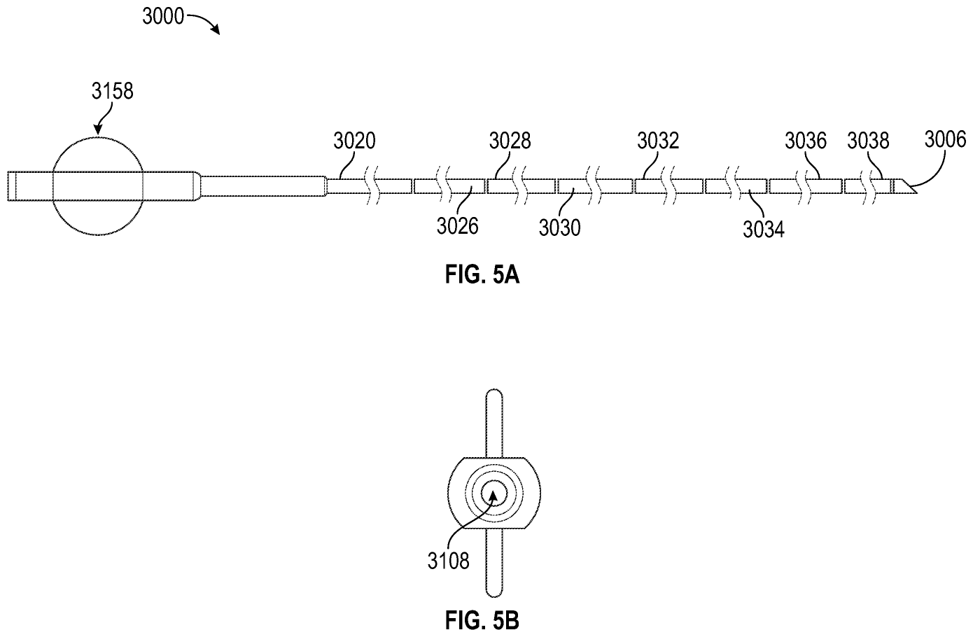

For example, the marker band in FIG. 4E (with optional compression gaps omitted for simplicity) includes a tension element in the form of a plurality of intersecting struts 3166 defining a tubular body having a plurality of sidewall openings 3168, which may progressively increase or decrease in compressibility in the proximal direction. The marker band and associated tension element struts 3166 may be slip fit over the tie layer with the coil wrapped around the outside of at least a portion of the length of the tension elements, with or without application of an adhesive prior to wrapping the coil. Alternatively, a plurality of proximal apexes may be formed in alignment on a transverse plane or other geometry that is complementary to the geometry of the distal end of the support structure (e.g., coil) in the catheter shaft, and be welded together end to end to provide a secure joint. Further, any of the embodiments of FIGS. 4A-4I may or may not include an axially extending slit, as described elsewhere herein to Referring to FIGS. 5A-5B, there is illustrated one example of an outer jacket segment stacking pattern for a progressive flexibility catheter of the type discussed in connection with FIG. 2. A distal segment 3038 may have a length within the range of about 1-3 cm, and a durometer of less than about 35D or 30D. An adjacent proximal segment 3036 may have a length within the range of about 4-6 cm, and a durometer of less than about 35D or 30D. An adjacent proximal segment 3034 may have a length within the range of about 4-6 cm, and a durometer of about 35D or less. An adjacent proximal segment 3032 may have a length within the range of about 1-3 cm, and a durometer within the range of from about 35D to about 45D (e.g., 40D). An adjacent proximal segment 3030 may have a length within the range of about 1-3 cm, and a durometer within the range of from about 50D to about 60D (e.g., about 55D). An adjacent proximal segment 3028 may have a length within the range of about 1-3 cm, and a durometer within the range of from about 35D to about 50D to about 60D (e.g., about 55D). An adjacent proximal segment 3026 may have a length within the range of about 1-3 cm, and a durometer of at least about 60D and typically less than about 75D. More proximal segments may have a durometer of at least about 65D or 70D. The distal most two or three segments may comprise a material such as Tecothane, and more proximal segments may comprise PEBAX or other catheter jacket materials known in the art. At least three or five or seven or nine or more discrete segments may be utilized, having a change in durometer between highest and lowest along the length of the catheter shaft of at least about 10D, preferably at least about 20D and in some implementations at least about 30D or 40D or more.

### Example Embodiments

A neurovascular catheter with enhanced tensile strength comprising one or more of the following:

an elongate flexible tubular body, having a proximal end, a distal end, and a side wall defining a central lumen;

a radiopaque marker adjacent the distal end, extending at least part way around a circumference of the tubular body; and

a tensile support extending axially in the side wall;

wherein the tensile support extends distally along a first side of the radiopaque marker, folds around a distal edge of the radiopaque marker, and extends along a second side of the radiopaque marker.

A neurovascular catheter of any example described herein, wherein the tensile support comprises a plurality of fibers.

A neurovascular catheter of any example described herein, wherein the tensile support comprises Vectran multifilament liquid crystal polymer fiber.

A neurovascular catheter of any example described herein, wherein the tensile support extends distally along a radially inwardly facing surface of the marker, around a distal end of the marker, and circumferentially around a radially outwardly facing surface of the marker.

A neurovascular catheter of any example described herein, wherein the tensile support extends circumferentially at least about 180 degrees around the marker.

A neurovascular catheter of any example described herein, wherein the radiopaque marker comprises a proximal face and a distal face, and the distal face inclines at an angle within a range of from about 45 degrees to about 80 degrees relative to the longitudinal axis of the central lumen.

A neurovascular catheter of any example described herein, comprising a distal port having an elliptical opening, and the elliptical opening comprises an area that is at least about 105% of a transverse cross-sectional area of the central lumen.

A neurovascular catheter of any example described herein, wherein the area of the elliptical opening is at least about 110% of the cross-sectional area of the central lumen.

A neurovascular catheter of any example described herein, wherein the elliptical opening inclines at an angle within a range of from about 55 degrees to about 65 degrees relative to the longitudinal axis of the central lumen.

A neurovascular catheter of any example described herein, wherein the proximal face on the radiopaque marker is approximately perpendicular to the longitudinal axis.

A neurovascular catheter of any example described herein, wherein the distal end is spaced apart from the distal face of the radiopaque marker to form an advance segment of the tubular body.

A neurovascular catheter of any example described herein, wherein the advance segment has an axial length within a range of from about 0.1 mm to about 5 mm.

A neurovascular catheter of any example described herein, wherein the axial length of the advance segment on a leading edge side of the tubular body is greater than the axial length of the advance segment on a trailing edge side of the tubular body.

A neurovascular catheter of any example described herein, wherein the radiopaque marker comprises an annular ring with at least one axial slit.

A neurovascular catheter of any example described herein, further comprising an inner liner, wherein the tensile support extends axially between the helical coil and the inner liner.

A neurovascular catheter of any example described herein, configured to withstand at least about 3.5 pounds tension before failure.

A neurovascular catheter of any example described herein, configured to withstand at least about 5 pounds tension before failure.

A neurovascular catheter of any example described herein, configured to withstand at least about 7 pounds tension before failure.

A neurovascular catheter of any example described herein, wherein the side wall includes an outer jacket comprising a plurality of tubular segments, a proximal tubular segment of the plurality of tubular segments having a durometer of at least about 60D, and a distal tubular segment of the plurality of tubular segments having a durometer of at most about 35D.

A neurovascular catheter comprising one or more of the following:

an elongate flexible tubular body, having a proximal end, an inclined distal end and a side wall defining a central lumen;

a distal leading tip on a first side of the inclined distal end; and

a preset curve in a distal zone of the tubular body;

wherein the distal leading tip lies on a concave side of the curve.

A neurovascular catheter of any example described herein, further comprising a tubular radiopaque marker embedded in the side wall, the tubular radiopaque marker comprising a proximal face and a distal face, wherein the distal face of the radiopaque marker inclines at an angle within a range of from about 45 degrees to about 80 degrees relative to the longitudinal axis of the central lumen.

A neurovascular catheter of any example described herein, comprising a distal port having an elliptical opening, and wherein the elliptical opening comprises an area that is at least about 105% of a cross-sectional area of the central lumen.

A neurovascular catheter of any example described herein, wherein the area of the elliptical opening is at least about 110% of the cross-sectional area of the central lumen.

A neurovascular catheter of any example described herein, wherein the area of the elliptical opening is within the range of from about 110% to about 125% of the cross-sectional area of the central lumen.

A neurovascular catheter of any example described herein, wherein the elliptical opening inclines at an angle within a range of from about 55 degrees to about 65 degrees relative to the longitudinal axis of the central lumen.

A neurovascular catheter of any example described herein, wherein the distal face of the radiopaque marker inclines at the angle within the range of from about 55 degrees to about 65 degrees relative to the longitudinal axis of the central lumen.

A neurovascular catheter of any example described herein, wherein the proximal face on the radiopaque marker is approximately perpendicular to the longitudinal axis.

A neurovascular catheter of any example described herein, wherein the distal end is spaced apart from the distal face of the radiopaque marker to form an advance segment of the tubular body.

A neurovascular catheter of any example described herein, wherein the advance segment has an axial length within a range of from about 0.1 mm to about 5 mm.

A neurovascular catheter of any example described herein, wherein the axial length of the advance segment on a leading edge side of the tubular body is greater than the axial length of the advance segment on a trailing edge side of the tubular body.

A neurovascular catheter of any example described herein, wherein the axial length of the advance segment on the leading edge side of the tubular body is at least about 20% longer than the axial length of the advance segment on the trailing edge side of the tubular body.

A neurovascular catheter of any example described herein, wherein the radiopaque marker comprises at least one axial slit.

A neurovascular catheter of any example described herein, further comprising a tensile support for increasing the tension resistance in the distal zone.

A neurovascular catheter of any example described herein, wherein the tensile support comprises an axially extending filament.

A neurovascular catheter of any example described herein, wherein the axially extending filament is carried between an inner liner and the helical coil.

A neurovascular catheter of any example described herein, wherein the axially extending filament increases the tensile strength of the tubular body to at least about 2 pounds.

A neurovascular catheter comprising one or more of the following:

an elongate flexible tubular body, having a proximal end, a distal zone and a side wall defining a central lumen;

a tubular radiopaque marker band embedded in the side wall in the distal zone;

wherein the radiopaque marker band has a first axial length measured along the side wall at a first circumferential position, and a second, longer axial length measured along the side wall at a second circumferential position offset around the circumference of the catheter by about 180 degrees from the first position; and

wherein the tubular body has a preset curve in the distal zone.

A neurovascular catheter of any example described herein, wherein the preset curve has a concave side and a convex side, and the second, longer axial length side of the marker is on the concave side of the curve.

A neurovascular catheter comprising one or more of the following:

an elongate flexible tubular body, having a proximal end, an inclined distal end and a side wall defining a central lumen;

a preset curve in a distal zone of the tubular body, having a convex side and a concave side; and

a filament extending axially along the sidewall of the distal zone;

wherein the distal leading tip lies on the concave side of the curve and the filament lies on the convex side of the curve.

A neurovascular catheter comprising one or more of the following:

an elongate flexible tubular body, having a proximal end, an inclined distal end and a side wall defining a central lumen;

a distal leading tip on a first side of the inclined distal end; and

a preset curve in a distal zone of the tubular body;

wherein the distal leading tip lies on a concave side of the curve.

A neurovascular catheter of any example described herein, further comprising a tubular radiopaque marker embedded in the side wall, the tubular radiopaque marker comprising a proximal face and a distal face, wherein the distal face of the radiopaque marker inclines at an angle within a range of from about 45 degrees to about 80 degrees relative to the longitudinal axis of the central lumen.

A neurovascular catheter of any example described herein, comprising a distal port having an elliptical opening, and wherein the elliptical opening comprises an area that is at least about 105% of a cross-sectional area of the central lumen.

A neurovascular catheter of any example described herein, wherein the area of the elliptical opening is at least about 110% of the cross-sectional area of the central lumen.

A neurovascular catheter of any example described herein, wherein the area of the elliptical opening is within the range of from about 110% to about 125% of the cross-sectional area of the central lumen.

A neurovascular catheter of any example described herein, wherein the elliptical opening inclines at an angle within a range of from about 55 degrees to about 65 degrees relative to the longitudinal axis of the central lumen.

A neurovascular catheter of any example described herein, wherein the distal face of the radiopaque marker inclines at the angle within the range of from about 55 degrees to about 65 degrees relative to the longitudinal axis of the central lumen.

A neurovascular catheter of any example described herein, wherein the proximal face on the radiopaque marker is approximately perpendicular to the longitudinal axis.

A neurovascular catheter of any example described herein, wherein the distal end is spaced apart from the distal face of the radiopaque marker to form an advance segment of the tubular body.

A neurovascular catheter of any example described herein, wherein the advance segment has an axial length within a range of from about 0.1 mm to about 5 mm.

A neurovascular catheter of any example described herein, wherein the axial length of the advance segment on a leading edge side of the tubular body is greater than the axial length of the advance segment on a trailing edge side of the tubular body.

A neurovascular catheter of any example described herein, wherein the axial length of the advance segment on the leading edge side of the tubular body is at least about 20% longer than the axial length of the advance segment on the trailing edge side of the tubular body.

A neurovascular catheter of any example described herein, wherein the radiopaque marker comprises at least one axial slit.

A neurovascular catheter of any example described herein, further comprising a tensile support for increasing the tension resistance in the distal zone.

An enhanced flexibility neurovascular catheter of any example described herein, wherein the tensile support comprises an axially extending filament.

A neurovascular catheter of any example described herein, wherein the axially extending filament is carried between an inner liner and the helical coil.

A neurovascular catheter of any example described herein, wherein the axially extending filament increases the tensile strength of the tubular body to at least about 2 pounds.

A neurovascular catheter comprising one or more of the following:

an elongate flexible tubular body, having a proximal end, a distal zone and a side wall defining a central lumen;

a tubular radiopaque marker band embedded in the side wall in the distal zone;

wherein the radiopaque marker band has a first axial length measured along the side wall at a first circumferential position, and a second, longer axial length measured along the side wall at a second circumferential position offset around the circumference of the catheter by about 180 degrees from the first position; and

wherein the tubular body has a preset curve in the distal zone.

A neurovascular catheter of any example described herein, wherein the preset curve has a concave side and a convex side, and the second, longer axial length side of the marker is on the concave side of the curve.

A neurovascular catheter comprising one or more of the following:

an elongate flexible tubular body, having a proximal end, an inclined distal end and a side wall defining a central lumen;

a preset curve in a distal zone of the tubular body, having a convex side and a concave side; and

a filament extending axially along the sidewall of the distal zone;

wherein the distal leading tip lies on the concave side of the curve and the filament lies on the convex side of the curve.

What is claimed is:

1. A neurovascular catheter, comprising:
an elongate flexible tubular body, having a proximal end, an inclined distal end and a side wall defining a central lumen;
a distal leading tip on a first side of the inclined distal end; and
a preset curve in a distal zone of the tubular body;
wherein the distal leading tip lies on a concave side of the curve.

2. A neurovascular catheter as in claim 1, further comprising a tubular radiopaque marker embedded in the side wall, the tubular radiopaque marker comprising a proximal face and a distal face, wherein the distal face of the radiopaque marker inclines at an angle within a range of from about 45 degrees to about 80 degrees relative to a longitudinal axis of the central lumen.

3. A neurovascular catheter as in claim 1, comprising a distal port having an elliptical opening, and wherein the elliptical opening comprises an area that is at least about 105% of a cross-sectional area of the central lumen.

4. A neurovascular catheter as in claim 3, wherein the area of the elliptical opening is at least about 110% of the cross-sectional area of the central lumen.

5. A neurovascular catheter as in claim 4, wherein the area of the elliptical opening is within a range of from about 110% to about 125% of the cross-sectional area of the central lumen.

6. A neurovascular catheter as in claim 3, wherein the elliptical opening inclines at an angle within a range of from about 55 degrees to about 65 degrees relative to a longitudinal axis of the central lumen.

7. A neurovascular catheter as in claim 2, wherein the distal face of the radiopaque marker inclines at the angle within the range of from about 55 degrees to about 65 degrees relative to the longitudinal axis of the central lumen.

8. A neurovascular catheter as in claim 2, wherein the proximal face on the radiopaque marker is approximately perpendicular to the longitudinal axis.

9. A neurovascular catheter as in claim 2, wherein the distal end is spaced apart from the distal face of the radiopaque marker to form an advance segment of the tubular body.

10. A neurovascular catheter as in claim 9, wherein the advance segment has an axial length within a range of from about 0.1 mm to about 5 mm.

11. A neurovascular catheter as in claim 9, wherein an axial length of the advance segment on a leading edge side of the tubular body is greater than the axial length of the advance segment on a trailing edge side of the tubular body.

12. A neurovascular catheter as in claim 11, wherein the axial length of the advance segment on the leading edge side of the tubular body is at least about 20% longer than the axial length of the advance segment on the trailing edge side of the tubular body.

13. A neurovascular catheter as in claim 2, wherein the radiopaque marker comprises at least one axial slit.

14. A neurovascular catheter as in claim 1, further comprising a tensile support for increasing a tension resistance in the distal zone.

15. A neurovascular catheter as in claim 14, wherein the tensile support comprises an axially extending filament.

16. A neurovascular catheter as in claim 15, wherein the axially extending filament is carried between an inner liner and a helical coil.

17. A neurovascular catheter as in claim 15, wherein the axially extending filament increases a tensile strength of the tubular body to at least about 2 pounds.

18. A neurovascular catheter, comprising:
an elongate flexible tubular body, having a proximal end, a distal zone and a side wall defining a central lumen; and
a tubular radiopaque marker band embedded in the side wall in the distal zone;
wherein the radiopaque marker band has a first axial length measured along the side wall at a first circumferential position, and a second, longer axial length measured along the side wall at a second circumferential position offset around a circumference of the catheter by about 180 degrees from the first position; and
wherein the tubular body has a preset curve in the distal zone.

19. A neurovascular catheter as in claim 18, wherein the preset curve has a concave side and a convex side, and the second, longer axial length side of the marker is on the concave side of the curve.

20. A neurovascular catheter, comprising:
an elongate flexible tubular body, having a proximal end, an inclined distal end and a side wall defining a central lumen;
a preset curve in a distal zone of the tubular body, having a convex side and a concave side; and
a filament extending axially along a sidewall of the distal zone;
wherein a distal leading tip lies on the concave side of the curve and the filament lies on the convex side of the curve.
